(19) United States
(12) Patent Application Publication
(10) Pub. No.: US 2016/0074320 A1
(43) Pub. Date: Mar. 17, 2016

(54) PHARMACEUTICAL COMPOSTIONS COMPRISING KISSPEPTIN OR DERIVATIVES THEREOF

(71) Applicants: OURO FINO SAÚDE ANIMAL LTDA, Cravinhos (BR); UNIVERSIDADE DE SAO PAULO - USP, São Paulo (BR)

(72) Inventors: Dolivar Coraucci NETO, Sertãozinho (BR); Gustavo Guerino MACEDO, Sao Paulo (BR); Pietro Sampaio BARUSELLI, Sao Paulo (BR)

(73) Assignees: UNIVERSIDADE DE SAO PAULO - USP, Sao Paulo (BR); OURO FINO SAUDE ANIMAL LTDA, Cravinhos (BR)

(21) Appl. No.: 14/783,476

(22) PCT Filed: Apr. 9, 2014
(86) PCT No.: PCT/BR2014/000117

§ 371 Date: Oct. 9, 2015

(30) Foreign Application Priority Data
Apr. 12, 2013 (BR) 10 2013 0089907

(51) Int. Cl. A61K 9/00; A61K 47/14; A61K 47/18; A61K 47/12; A61K 47/44; A61K 47/22; A61K 47/34; A61K 47/10; A61K 38/09; A61K 47/26

(52) U.S. Cl. CPC A61K 9/0019; A61K 38/09; A61K 47/14; A61K 47/183; A61K 47/26; A61K 47/44; A61K 47/22; A61K 47/34; A61K 9/0024; A61K 47/10; A61K 47/12

(57) ABSTRACT

The present invention relates to pharmaceutical compositions comprising a peptide that stimulates the release of gonadotropins and sexual steroids. More specifically, the present invention provides pharmaceutical compositions comprising kisspeptin, preferably in the kp-10 form, or derivatives thereof, for use in ovulation cycle inducing and/or infertility treatment programs. The formulations according to the present invention belong to two main groups: injectable solutions and implantable formulations. The injectable solutions according to the present invention can be divided into immediate release solutions and prolonged action solutions. The implantable formulations according to the present invention can be prepared using an RTV silicone elastomer, a rapid vulcanization silicone elastomer or a rapid vulcanization silicone elastomer with a release modulator.

### FIELD OF THE INVENTION

The present invention refers to pharmaceutical compositions comprising a peptide that stimulates the release of gonadotropins. More specifically, the present invention provides pharmaceutical compositions comprising kisspeptin or derivatives thereof, for use in the induction of cyclicity and/or treatment of infertility.

### BACKGROUND OF THE INVENTION

It is understood by kisspeptin the hydrophobic protein of 145 amino acids, encoded by suppressor gene of Kiss-1 cancer. This protein can be cleaved in smaller fragments, giving rise to forms Kp-10, Kp-13, Kp-14 and Kp-54, wherein the number presented after “Kp” indicates the size of the resulting amino acids sequence. First of them, Kp-10, is the most commonly found and the one with less variation between species.

There are numerous studies about the activity of this protein and its role in reproduction. The first evidence of a link between Kp and reproduction were observed by means of genetic analyzes in humans and rodents, which revealed mutations causing the loss of functionality of encoding gene of Kiss-1 receptor, which were associated with delayed onset of puberty and hypogonadism-hypogonadotrophic, caused by deficiency of gonadotropins releasing hormone (GnRH). This finding, that a simple receiver has such a profound effect at puberty, intensified research in the pursuit of understanding how is the activation of the hypothalamic-hypophyseal-gonadal (HHG) axis and the onset of puberty.

Kisspeptin proved to be of great importance to the onset of puberty (in both humans and other animals) and a single injection is capable of generating considerable increase in secretion of gonadotropins, which are glycoprotein hormones secreted by gonadotropic cells of hypophysis in vertebrates. There are two types of gonadotropic hormones, Luteinizing Hormone (LH) and Follicle Stimulating Hormone (FSH), both having as acting target the ovaries and the production of steroid hormones. Kisspeptin acts in the Hypothalamus-Hypophyseal-Gonadal by stimulus of GnRH (Gonadotropins Releasing Hormone, produced by hypothalamus), which in turn stimulates hypophysis to produce the gonadotropins. It also has direct action in the genital tract organs and yet it is suggested direct action in the hypophysis. The discovery of the involvement of kisspeptin in controlling reproduction allowed extrapolating the mechanism for industrial application.

Currently it is well accepted that the Kp is the most potent stimulator of release of GnRH. Small quantities (pmol) of Kp-10 has a powerful capacity to release GnRH/gonadotropins, especially luteinizing hormone (LH). In prepubertal heifers, the IV administration of KP-10 induces the release of LH and growth hormone (GH). In the same sense, Redmond and collaborators noted that prepubertal heifers are responsive to IV injections of Kp-10, resulting in the increase of frequency and amplitude of the pulses of LH s well as its average concentration. As a consequence, the sheep of Kp-10 treatment presented LH pre-ovulatory peak, with consequent ovulation, as opposed to the animals of control group (saline). This data indicates that the neurons activation containing Kp is involved in the pubertal maturation of HHG axis.

The potent release of LH in response to administration of Kp indicates that during the follicular phase, Kp could promote a way of synchronizing ovulation, thereby increasing the efficiency of artificial insemination. The initial response to Kp-10 administration is a continuous and rapid increase in the plasma concentration of estradiol (E₂) and a synchronized peak of LH after 2 h. This is shown by a series of experiments of Caraty et al. (2007) in sheep, where the peripheral infusion of Kp-10 reproduced clearly the hormone changes usually observed during the end of the follicular phase of estrous cycle, by stimulating the secretion of follicular E₂ and its latent negative and positive feedback in the secretion of LH.

In this study it was applied Kp-10 by 30 h after the removal of intra-vaginal device of progesterone (P₄) (CIDR®), e it was observed that the peak of LH ran more quickly and synchronized after the onset of the infusion of Kp-10 (nearly 32 h after removal of CIDR®), that in the group without treatment where there was large dispersion in the animals, with a variation of the peak of LH from 42 h to more than 65 h after removing the P₄. Furthermore, the elevation of plasma concentration of E₂ is much faster in the first situation. This may be because of Kp-10 also stimulates the release of FSH, which under normal physiological conditions, it would be decreasing in the follicular phase. Another factor that may contribute is the Kiss-1 gene expression and the Kiss-1r receptor in the granulous cells, suggesting that kp-10 can be involved with the steroidogenesis. This way, it is possible that the peripheral treatment with Kp-10 has not only central action, in the increased secretion of gonadotropins, but also ovarian stimulating the secretion of E₂, leading to a rapid onset of peak of LH. Although the shortening of the follicular phase can cause issues arise about the quality of ovulatory follicles and their ovocytes, an ability of Kp to accelerate follicle growth and shortening the ovulation time in ovine, offer an interesting alternative in the fertility control of bovines.

For the restoration of cyclicality of animals in anestrus, Caraty et al used sheep as a model and administered low and sustained (30 to 48 h) doses IV of Kp-10 and noted ovulation in more than 80% of animals.

The demonstration that the use of Kp can stimulate the final maturation of the dominant follicle, synchronize surge of LH and ovulation in cyclic animals or in deep anestrus allows the therapeutic application and use of Kp in programs of estrus synchronization in animals, enabling the development of new products heretofore not known.

Patents WO200285399 and WO2004060264 from Takeda Chemical Industries disclose preparations containing metastin able to inhibit metastasis in cancer and a derivative of metastin, pharmaceutical product, method, use and agents containing the derivative with various actions, including preventing and treating cancers, respectively.

Patent WO2004101747 from General Hospital claims compounds for diagnosis and treatment of reproductive disorders or other, wherein he suppression of gonadal steroids has benefits. The identified compounds can be used as contraception or in the treatment of infertility, as in IVF. It also describes GPR54 mutant molecules and polypeptides as well as its use.

Patent WO2004063221 from Takeda Chemical Industries discloses a derivative of metastin, pharmaceutical product, method, use and agent containing the derivative with several actions, including inducing or stimulating ovulation.

Patent EP1604682 from Takeda Chemical Industries discloses derivatives of metastin, pharmaceutical product, method, use and agent containing the derivative to induce or stimulate ovulation.

Patent WO2005117939 from Applied Research Systems protects a method and compositions for treatment of infertility in mammals, uses of derivatives of Kiss-1 protein.

Patent EP1464652 from Inserm-Institut Nacional de La Sante et de La. Recherche medicale, Universite Paris Descartes, Universite Paris Sud, protects a GPR54 receptor agonist or antagonist for the treatment of disorders related to the gonadotropin and compositions comprising GnRH and Kiss-1 agonist.

The programs of ovulation synchronization in production animals consist in the use of hormonal treatments to induce a greater number of females to heat/ovulation on a given period of time. There are vast choices of treatments available on the market today, comprising the use of estrogens, F2α prostaglandin or analogues, GnRH injections, eCG, cloprostenol, detection of corpus luteum in the ovary, among others. However, some have high cost, require much effort and do not get satisfactory results.

The present invention seeks to solve the problems presented by means of an innovative pharmaceutical composition containing kisspeptin and derivatives thereof for use in the programs of cyclicity induction and/or treatment of infertility.

The formulations of the present invention are comprised of two main groups: injectable solutions and implantable formulations.

The injectable solutions are characterized by the route of administration (parenteral). They are normally used when a quick response is required, when the active ingredient is inactivated by other route of administration or when the drug disgusts the patient. The rate of release of the active ingredient can be modified by the addition of excipients, the change of oily vehicle and the variation in the manufacturing process. For example, the more viscous oily solutions tend to increase the duration of drug release. The addition of polymeric agents, in turn, can provide greater control of drug release, sustain its therapeutic action over time and/or release the drug to the level of a given tissue or target organ.

The implantable formulations are characterized by systems of drug release intended for insertion, invasively, in tissues and organs. The advantages of this type of formulation consist in avoiding damage to skin and tissue (sometimes associated with injections), causing less stress to the animal at the time of administration and allow full control in the release of the drug, which can be interrupted at any time, characteristics which favor the use of this type of formulation in breeding protocols in animals in the agricultural sector.

### DESCRIPTION OF THE INVENTION

The injectable solutions of the present invention can be separated into immediate release solutions (Example 1) and long-acting solutions (Example 2). Both the immediate release solutions and the long-acting solutions have a concentration of 0.01 to 30.0% of the active ingredient kisspeptin or derivatives thereof. Preferably, the kp-10 form or derivatives thereof.

Pharmaceutically acceptable excipients which may be used in the manufacture of immediate release injectable solutions of the present invention include various organic or inorganic substances conventionally used as materials for pharmaceutical preparations. Such substances include, for example, a solubilizer, a buffering agent and a vehicle. Furthermore, conventional additives such as a preservative, a chelating and/or an anti-oxidant agent are appropriately used in suitable amounts.

Examples of solubilizers include polysorbates, lecithin, poloxamer, polyoxyethylene alkyl ethers, derivatives of castor oil polyoxyethylene, polyoxyethylene stearates, pyrrolidone, sodium lauryl sulfate, propylene glycol, glycerin, triacetin, sorbitol, cyclomethicone, polydextrose, medium and long chain esters or triglycerides, sorbitan polyethylene glycol esters, mannitol esters, preferably propylene glycol in concentrations of 1.0 to 10.0% solution.

Examples of buffering agents include solution of glycine and hydrochloric acid, citric acid and sodium citrate, citric acid and dibasic sodium phosphate, monobasic sodium phosphate and dibasic sodium phosphate, sodium acetate and acetic acid, preferably sodium acetate and acetic acid in the following concentrations: 0.5 to 1.0% and 0.1 to 1.0% solution, respectively.

Examples of vehicles preferably include water up to 100% solution.

Examples of preservatives include methylparaben (Nipagin), propylparaben (Nipasol), benzyl alcohol, parabens, benzalkonium chloride, bronopol, cetrimide, chlorobutanol, phenoxyethanol, imidazolidinyl urea, isothiazolinone, benzoic and sorbic acids and derivatives thereof, dehydroacetic acid, ferulic acid. Preferably, methylparaben (Nipagin) and propylparaben (Nipasol) at the following concentrations: 0.1 to 10.0% and 0.01 to 0.1% solution, respectively.

Examples of chelating and/or anti-oxidant agents include: BHT, BHA, disodium and tetrasodium EDTA, propyl gallate, sodium metabisulfite, tocopherols, phenolic acids, ascorbic acid and derivatives thereof and citric acid. Preferably, disodium EDTA in concentrations of 0.010 to 0.100% solution.

Pharmaceutically acceptable excipients which may be used in the manufacture of long-acting injectable solutions of the present invention include various organic or inorganic substances conventionally used as materials for pharmaceutical preparations. Such substances include, for example, a surfactant, a biocompatible oil, polymeric agents, an anti-oxidant and a vehicle.

Examples of surfactants include sorbitan esters (sorbitan trilaurate. sorbitan monopalmitate, sorbitan monostearate, sorbitan tristearate, sorbitan monooleate, sorbitan trioleate), mannitol esters ou lecithins, preferably sorbitan monooleate in concentrations of 0.1 to 10.0% solution.

Examples of biocompatible oils include castor oil, canola oil, corn oil, cottonseed oil, olive oil, peanut oil, sesame oil, soybean oil, cottonseed oil, grape seed oil, sunflower oil, preferably castor oil in concentrations of 10.0% until completing 100.0% solution.

Examples of polymeric agents include pemullen TR2, polymeric emulsifiers of polyacrylic acid and chitosan, preferably pemullen TR2 in concentrations of 0.1 to 1.0% solution.

Examples of anti-oxidant agents include DL-α-tocopherol, BHA, BHT, disodium and tetrasodium EDTA, propyl gallate, sodium metabisulfite, tocopherols, phenolic acids, ascorbic acid and derivatives thereof, citric acid, preferably DL-α-tocopherol in concentrations of 0.01 to 0.1% solution.

Examples of vehicles include GTCC crodamol, hydrocarbons of low viscosity or mineral oils (liquid paraffin), or fatty acid esters of 6 to 18 carbon atoms, or fatty acid esters of propylene glycol or vegetable oils (castor oil, corn oil, peanut oil, sesame oil, olive oil, palm oil, soybean oil, cottonseed oil, grape seed oil, sunflower oil) or medium chain triglycerides (triglyceride of capric/caprylic acid), preferably GTCC crodamol in up to 100% solution.

The implantable formulations of the present invention can be separated in implantable solutions presenting the use of RTV silicone elastomer (room temperature vulcanization) (see Example 3), the use of rapid vulcanization silicone elastomer (high temperature) (see Example 4) and the use of rapid vulcanization silicone elastomer with release modulator (see Example 5). All implantable formulations have a concentration of 0.01 to 30.0% of the active ingredient kisspeptin or derivatives thereof. Preferably, the form kp-10 or derivatives thereof.

Pharmaceutically acceptable excipients which may be used in the manufacture of implantable formulations of the present invention include various organic or inorganic substances conventionally used as materials for pharmaceutical preparations. These substances include, for example, a catalyst (curing agent) and a RTV silicone elastomer according to Example 3, in the following concentrations: 1 to 10% and up to 100% of the implantable formulation, respectively.

Examples of silicone elastomer preferably include the RTV600 silicones from Momentive Raw Material®.

The organic or inorganic carrier substances conventionally used as materials for pharmaceutical preparations include, for example, a catalyst (curing agent) and a silicone elastomer according to Example 4, in the following concentrations: 1 to 10% and up to 100% of the implantable formulation, respectively.

Examples of silicone elastomer of Example 4 preferably include the Tufel II silicones, Ruber 94006 silicone from Momentive Raw Material®.

The organic or inorganic carrier substances conventionally used as materials for pharmaceutical preparations include, for example, a compound and a silicone elastomer according to Example 5 in the following concentrations: 1 to 50% and up to 100% of the implantable formulation, respectively.

Examples of silicone elastomer of Example 5 preferably include the LSR 265, LSR 2050 or Silopren LSR 2650 silicones from Momentive Raw Material®.

### EXAMPLES

### Example 1

### Injectable Solution

| COMPONENTS | | | |
|---|---|---|---|
| | RAW | | |
| FUNC- | MATE- | CONCEN- | POSSIBLE |
| TION | RIALS | TRATION | SUBSTITUENTS |
| Active | Kisseptin | 0.01-30.0% | — |
| | (kp-10) or | | |
| | derivative | | |
| Solubi- | Propylene | 10-10.0% | polysorbates, lecithin, |
| lizer | glycol | | poloxamer, polyoxyethylene |
| | | | alkyl ethers, derivatives of |
| | | | castor oil polyoxyethylene, |
| | | | polyoxyethylene stearates, |
| | | | pyrrolidone, sodium lauryl |
| | | | sulfate, propylene glycol, |
| | | | glycerin, triacetin, sorbitol, |
| | | | cyclomethicone, |
| | | | polydexfrose, medium and |
| | | | long chain esters or |
| | | | triglycerides, sorbitan |
| | | | polyethylene glycol esters, |
| | | | mannitol esters |
| Chelating/ | Disodium | 0.010-0.100% | BHT, BHA, disodium and |
| anti- | EDTA | | tetrasodium EDTA, propyl |
| oxidant | | | gallate, sodium metabisulfite, |
| agents | | | tocopherols, phenolic acids, |
| | | | ascorbic acid and derivatives |
| | | | thereof and citric acid |
| Preserva- | Methyl- | 0.100-1.00%/ | benzyl alcohol, parabens, |
| tive | paraben | 0.010-0.100% | benzalkonium chloride, |
| | (niagin) | | bronopol, cetrimide, |
| | Propyl- | | chlorobutanol, |
| | paraben | | phenoxyethanol, |
| | (nipasol) | | imidazolidinyl urea, |
| | | | isothiazolinone, benzoic, |
| | | | sorbic acids and derivatives, |
| | | | dehydroacetic acid, ferulic |
| | | | acid |
| Buffering | Sodium | 0.5-1.0%/ | solution of glycine and |
| agents | acetate/ | 0.1-1.0% | hydrochloric acid, citric acid |
| | acetic | | and sodium citrate, citric acid |
| | acid | | and dibasic sodium |
| | | | phosphate, monobasic |
| | | | sodium phosphate and |
| | | | dibasic sodium phosphate, |
| | | | sodium acetate and acetic |
| | | | acid |
| Vehicle | Reverse | q.s. 100% | — |
| | osmosis | | |
| | water | | |

### Example 2

### Long-Acting Injectable Solution

| COMPONENTS | | | |
|---|---|---|---|
| | RAW | | |
| FUNC- | MATE- | CONCEN- | POSSIBLE |
| TION | RIALS | TRATION | SUBSTITUENTS |
| Active | Kisseptin | 0.01-30.0% | — |
| | (kp-10) or | | |
| | derivative | | |
| Surfactant | Sorbitan | 0.1-10.0% | sorbitan esters (sorbitan |
| | monooleate | | trilaurate, sorbitan |
| | | | monopalmitate, sorbitan |
| | | | monostearate, sorbitan |
| | | | tristearate, sorbitan |
| | | | monooleate, sorbitan |
| | | | trioleate), mannitol esters |
| | | | ou lecithins |
| Biocom- | Castor oil | 10.0-100.0% | canola oil, corn oil, |
| patible | | | cottonseed oil, olive oil, |
| oil | | | peanut oil, sesame oil, |
| | | | soybean oil, cottonseed oil, |
| | | | grape seed oil, |
| | | | sunflower oil |
| Polymeric | Pemullen | 0.1-1.0% | polymeric emulsifiers of |
| agents | TR2 | | polyacrylic acid |
| | | | and chitosan |
| Anti- | DL-α- | 0.01-0.1% | BHA, BHT, disodium and |
| oxidant | tocopherol | | tetrasodium EDTA, propyl |
| | | | gallate, sodium |
| | | | metabisulfite, |
| | | | tocopherols, phenolic acids, |
| | | | ascorbic acid and |
| | | | derivatives |
| | | | thereof, citric acid |
| Vehicle | GTCC | q.s. 100% | hydrocarbons of |
| | cromadol | | low viscosity or |
| | | | mineral oils (liquid |
| | | | paraffin), or fatty acid esters |
| | | | of 6 to 18 carbon atoms, or |
| | | | fatty acid esters of propylene |
| | | | glycol or vegetable oils |
| | | | (castor oil, corn oil, peanut |
| | | | oil, sesame oil, olive oil, palm |
| | | | oil, soybean oil, cottonseed |
| | | | oil, grape seed oil, sunflower |
| | | | oil) or medium chain |
| | | | triglycerides (triglyceride of |
| | | | capric/caprylic acid) |

### Example 3

### Implantable Formulations, Use of RTV Silicone Elastomer (Room Temperature Vulcanization)

| COMPONENTS | | | |
|---|---|---|---|
| | RAW | | |
| | MATE- | CONCEN- | |
| FUNCTION | RIALS | TRATION | POSSIBLE |
| Active | Kisspeptin | 0.01-30.0% | — |
| | (kp-10) or | | |
| | derivative | | |
| Catalyst | — | 1-10% | — |
| (curing agent) | | | |
| RTV silicone | — | q.s. 100% | RTV600 silicones |
| elastomer | | | from Momentive |
| | | | Raw Material ® |

### Example 4

### Implantable Formulations, Use of Rapid Vulcanization Silicone Elastomer (High Temperature)

| COMPONENTS | | | |
|---|---|---|---|
| | RAW | | |
| | MATE- | CONCEN- | |
| FUNCTION | RIALS | TRATION | POSSIBLE |
| Active | Kisspeptin | 0.01-30.0% | — |
| | (kp-10) or | | |
| | derivative | | |
| Catalyst | — | 1-10% | — |
| (curing agent) | | | |
| Silicone | — | q.s. 100% | Tufel II silicone, |
| elastomer | | | Ruber 94006 |
| | | | silicone from |
| | | | Momentive Raw |
| | | | Material ® |

### Example 5

### Implantable Formulations, Use of Rapid Vulcanization Silicone Elastomer (High Temperature) with Release Modulator

COMPONENTS

| | RAW | | |
|---|---|---|---|
| | MATE- | CONCEN- | POSSIBLE |
| FUNCTION | RIALS | TRATION | SUBSTITUENTS |
| Active | Kisspeptin | 0.01-30.0% | — |
| | (kp-10) or | | |
| | derivative | | |
| Silicone | — | q.s. 100% | LSR 265, LSR |
| elastomer | | | 2050 or Silopren |
| | | | LSR 2650 |
| | | | silicones from |
| | | | Momentive Raw |
| | | | Material ® |

1. Pharmaceutical compositions comprising kisspeptin or derivatives thereof, used in synchronization programs of production animal estrus, comprising a concentration of 0.01-30.0% Kp-10 or derivatives thereof.

2. Pharmaceutical compositions comprising kisspeptin or derivatives thereof, according to claim 1, wherein they are in the form of injectable solutions or implantable formulations.

3. Pharmaceutical compositions comprising kisspeptin or derivatives thereof, according to claim 2, wherein the injectable solutions are of immediate release and comprise a solubilizer, a buffering agent, a preservative, a chelating and/or an anti-oxidant agent and a vehicle.

4. Pharmaceutical compositions comprising kisspeptin or derivatives thereof, according to claim 3, wherein the solubilizers include: polysorbate, lecithin, poloxamer, polyoxyethylene alkyl ethers, derivatives of castor oil polyoxyethylene, polyoxyethylene stearates, pyrrolidone, sodium lauryl sulfate, propylene glycol, glycerin, triacetin, sorbitol, cyclomethicone, polydextrose, medium and long chain esters or triglycerides, polyethylene glycol sorbitan esters, propylene glycol esters of mannitol.

5. Pharmaceutical compositions comprising kisspeptin or derivatives thereof, according to claim 4, wherein the solubilizer preferably include the propylene glycol in concentrations of 1.0 to 10.0% solution.

6. Pharmaceutical compositions comprising kisspeptin or derivatives thereof, according to claim 3, wherein the buffering agents include solution of glycine and hydrochloric acid, citric acid and sodium citrate, citric acid and dibasic sodium phosphate, monobasic sodium phosphate and dibasic sodium phosphate, sodium acetate and acid.

7. Pharmaceutical compositions comprising kisspeptin or derivatives thereof, according to claim 6, wherein the buffering agent preferably include the sodium acetate in the following concentrations: 0.5 to 1.0% and 0.1 to 1.0% solution, respectively.

8. Pharmaceutical compositions comprising kisspeptin or derivatives thereof, according to claim 3, wherein the preservatives include: methylparaben (Nipagin), propylparaben (Nipasol), benzyl alcohol, parabens, benzalkonium chloride, bronopol, cetrimide, chlorobutanol, phenoxyethanol, imidazolidinyl urea, isothiazolinone, benzoic, sorbic acids and derivatives, dehydroacetic acid, ferulic acid.

9. Pharmaceutical compositions comprising kisspeptin or derivatives thereof, according to claim 8, wherein the preservatives preferably include methylparaben (Nipagin) and propylparaben (Nipasol) in the following concentrations: 0.1 a 1.00% and 0.010 to 0.1% solution, respectively.

10. Pharmaceutical compositions comprising kisspeptin or derivatives thereof, according to claim 3, wherein the chelating and/or anti-oxidant agents include BHT, BHA, disodium and tetrasodium EDTA, propyl gallate, sodium metabisulfite, tocopherols, phenolic acids, ascorbic acid and derivatives thereof and citric acid.

11. Pharmaceutical compositions comprising kisspeptin or derivatives thereof, according to claim 10, wherein the chelating and/or anti-oxidant agents preferably include, disodium EDTA in concentrations of 0.010 to 0.1% solution.

12. Pharmaceutical compositions comprising kisspeptin or derivatives thereof, according to claim 3, wherein the vehicles preferably include reverse osmosis water in q.s. (quantum satis) 100% of solution.

13. Pharmaceutical compositions comprising kisspeptin or derivatives thereof, according to claim 2, wherein the injectable solutions are of prolonged action and comprise a surfactant, a biocompatible oil, polymeric agents, an anti-oxidant and a vehicle.

14. Pharmaceutical compositions comprising kisspeptin or derivatives thereof, according to claim 13, wherein the surfactants include: sorbitan esters (sorbitan trilaurate, sorbitan monopalmitate, sorbitan monostearate, sorbitan tristearate, sorbitan monooleate, sorbitan trioleate), mannitol esters or lecithins.

15. Pharmaceutical compositions comprising kisspeptin or derivatives thereof, according to claim 14, wherein the surfactants preferably include sorbitan monooleate in concentrations of 0.1 to 10.0% solution.

16. Pharmaceutical compositions comprising kisspeptin or derivatives thereof, according to claim 13, wherein the biofuel oils include: castor oil, canola oil, corn oil, cottonseed oil, olive oil, peanut oil, sesame oil, soybean oil, cottonseed oil, grape seed oil, sunflower oil.

17. Pharmaceutical compositions comprising kisspeptin or derivatives thereof, according to claim 16, wherein the biocompatible oils preferably include castor oil in concentrations of 10.0 until completing 100.0% solution.

18. Pharmaceutical compositions comprising kisspeptin or derivatives thereof, according to claim 13, wherein the polymeric agents include: pemullen TR2, polymeric emulsifiers of polyacrylic acid and chitosan.

19. Pharmaceutical compositions comprising kisspeptin or derivatives thereof, according to claim 18, wherein the polymeric agents preferably include pemullen TR2 in concentrations of 0.1 to 1.0% solution.

20. Pharmaceutical compositions comprising kisspeptin or derivatives thereof, according to claim 13, wherein the anti-oxidant agents include: DL-α-tocopherol, BHA, BHT, disodium and tetrasodium EDTA, propyl gallate, sodium metabisulfite, tocopherols, phenolic acids, ascorbic acid and derivatives thereof and citric acid.

21. Pharmaceutical compositions comprising kisspeptin or derivatives thereof, according to claim 20, wherein the anti-oxidant agents preferably include DL-α-tocopherol in concentrations of 0.01 to 0.1% solution.

22. Pharmaceutical compositions comprising kisspeptin or derivatives thereof, according to claim 13, wherein the vehicles include: GTCC crodamol, hydrocarbons of low viscosity or mineral oils (liquid paraffin), or fatty acid esters of 6 to 18 carbon atoms, or fatty acid esters of propylene glycol or vegetable oils (castor oil, corn oil, peanut oil, sesame oil, olive oil, palm oil, soybean oil, cottonseed oil, grape seed oil, sunflower oil) or medium chain triglycerides (triglyceride of capric/caprylic acid).

23. Pharmaceutical compositions comprising kisspeptin or derivatives thereof, according to claim 22, wherein the vehicles preferably include crodamol GTCC in q.s. (quantum satis) 100% of the solution.

24. Pharmaceutical compositions comprising kisspeptin or derivatives thereof, according to claim 2, wherein the implantable formulations use a RTV silicone elastomer and comprise a catalyst and a RTV silicone elastomer in the following concentrations: 1 to 10% and q.s. (quantum satis) 100% of the implantable formulation, respectively.

25. Pharmaceutical compositions comprising kisspeptin or derivatives thereof, according to claim 24, wherein the implantable formulations with the use of RTV silicone elastomer comprise preferably the RTV600 silicones from Momentive Raw Material®.

26. Pharmaceutical compositions comprising kisspeptin or derivatives thereof, according to claim 2, wherein the implantable formulations use rapid vulcanization silicone elastomer and comprise a catalyst and a silicone elastomer in the following concentrations: 1 to 10% and q.s. (quantum satis) 100% of the implantable formulation, respectively.

27. Pharmaceutical compositions comprising kisspeptin or derivatives thereof, according to claim 26, wherein the implantable formulations use rapid vulcanization silicone elastomer and preferably comprise the Tufel II silicones and Ruber 94006 silicone from Momentive Raw Material®.

28. Pharmaceutical compositions comprising kisspeptin or derivatives thereof, according to claim 2, wherein the implantable formulations use rapid vulcanization silicone elastomer with release modulator and comprise a catalyst and a silicone elastomer in the following concentrations: 1 to 50% and q.s. (quantum satis) 100% of the implantable formulation, respectively.

29. Pharmaceutical compositions comprising kisspeptin or derivatives thereof, according to claim 28, wherein the implantable formulations use rapid vulcanization silicone elastomer with release modulator and preferably comprise dimethicone.

30. Pharmaceutical compositions comprising kisspeptin or derivatives thereof, according to claim 28, wherein das the implantable formulations use rapid vulcanization silicone elastomer with release modulator and preferably comprise LSR 265, LSR 2050 or Silopren LSR 2650 silicones from Momentive Raw Material®.
